(19) United States
(12) Patent Application Publication
(10) Pub. No.: US 2024/0110910 A1
(43) Pub. Date: Apr. 4, 2024

(54) RAPID TESTING MECHANISM AND METHOD FOR RESPIRATORY VIRAL PATHOGENS

(71) Applicant: Brandon Heeger, Avon, IN (US)

(72) Inventor: Brandon Heeger, Avon, IN (US)

(21) Appl. No.: 18/533,595

(22) Filed: Dec. 8, 2023

Related U.S. Application Data
(63) Continuation-in-part of application No. 17/224,076, filed on Apr. 6, 2021, now Pat. No. 11,644,456.
(60) Provisional application No. 63/016,335, filed on Apr. 28, 2020.

(51) Int. Cl. G01N 33/52

(52) U.S. Cl. CPC G01N 33/523

(57) ABSTRACT

A rapid testing mechanism for respiratory viral pathogens includes a filter material, which includes a pathogen binding adsorptive reagent, positioned to capture exhaled breath particles from a respiratory tract. When the exhaled breath particles pass through the filter material, the following occur: when the binding adsorptive reagent reacts, a positive test for respiratory viral pathogens is indicated by the filter material; and when the pathogen binding adsorptive reagent does not react, a negative test is indicated. A transparent membrane makes the positive or negative test indication visible through the external layer of the filter material and, thus, visible to the external environment. For privacy, however, a cover over the transparent membrane may be movable between open and closed positions.

### CROSS-REFERENCE TO RELATED APPLICATIONS

This patent application is a continuation-in-part of co-pending U.S. patent application Ser. No. 17/224,076, filed on Apr. 6, 2021, which claims the benefit of U.S. Provisional Patent Application Ser. No. 63/016,335, filed on Apr. 27, 2020; and is a continuation-in-part of co-pending U.S. patent application Ser. No. 17/895,807, filed on Aug. 25, 2022, which is a continuation-in-part of U.S. patent application Ser. No. 17/500,126, filed on Oct. 13, 2021 (which has issued as U.S. Pat. No. 11,454,625 on Sep. 27, 2022), which is a continuation of U.S. patent application Ser. No. 17/244,076, filed on Apr. 6, 2021, and claims the benefit of U.S. Provisional Patent Application Ser. No. 63/016,335, filed on Apr. 27, 2020; all of which are incorporated herein by reference.

### TECHNICAL FIELD

The present disclosure relates to the field of respiratory viral pathogen testing. More specifically, the present invention is directed to a rapid mechanism, in the form of a face mask, and method for respiratory viral pathogen testing, including an external visualization of test results.

### BACKGROUND

Conventionally, viral testing is aimed to identify a specific virus. In most situations, identifying a specific virus allows for the collection of epidemiological data and the opportunity for targeted treatment. For example, a patient diagnosed with a viral infection due to influenza might be provided with a prescription of antiviral medication.

However, for the vast majority of respiratory viruses, identifying the specific virus provides little benefit, as the treatment, including supportive care, does not change. As such, testing causes unnecessary cost and burden on the healthcare system. Similarly, there is currently little to no utility in screening asymptomatic individuals, outside of a pandemic or other unique situation.

In the setting of a pandemic, such as when a novel virus is involved, there is typically a lapse in development, production, and distribution of novel viral detection agents. This time delay allows for viral spread without epidemiologic data. Further complicating the scenario are asymptomatic carriers, such as with the recent COVID-19 pandemic. Identifying asymptomatic carriers has proven to be a unique challenge, and the lack of identification of asymptomatic carriers undoubtedly contributes to disease spread. For example, a person who is admitted to the hospital, without clinical evidence of a respiratory virus, might in fact be carrying, and spreading, the COVID-19 virus.

Due to testing limitations, including availability, cost, resource utilization, and time delay until test result, these individuals entering the hospital are typically not screened. They might be admitted to the hospital and spread the disease, unbeknownst to them and the numerous hospital employees they encounter. A similar scenario occurs even when cursory screening is deployed. For example, in the beginning of the COVID-19 pandemic people were being screened for the virus by answering a screening questionnaire and testing for the presence of a fever. This screening is low yield, especially when considering asymptomatic carriers. Innumerable scenarios such as the above could be described.

The present technology is directed to and addresses the issues identified above.

### SUMMARY

According to one aspect of the present disclosure, a rapid testing mechanism for respiratory viral pathogens includes a filter material positioned to capture exhaled breath particles from a respiratory tract of a mammal. At least a portion of the filter material includes a pathogen binding adsorptive reagent positioned between an internal layer and an external layer of the filter material. A transparent membrane is visible through the external layer of the filter material. When exhaled breath particles pass through the filter material, the following occur: when the binding adsorptive reagent reacts, a positive test for respiratory viral pathogens is indicated by the filter material; and when the pathogen binding adsorptive reagent does not react, a negative test for respiratory viral pathogens is individuated by the filter material. When the pathogen binding adsorptive reagent reacts, the positive test indication or the negative test indication are visible through the transparent membrane, which is exposed through the external layer.

### BRIEF DESCRIPTION OF THE DRAWINGS

FIG. 1 depicts a simplified view of a view mask, according to an exemplary embodiment of the present disclosure;

FIG. 2 depicts a simplified view of another face mask, indicating a positive test for respiratory viral pathogens;

FIG. 3 depicts a simplified view of another face mask, indicating a negative test for respiratory viral pathogens;

FIG. 4 depicts a simplified view of a face mask, according to an exemplary embodiment of the present disclosure, illustrating a negative test result visible through a transparent membrane;

FIG. 5 depicts a simplified view of the face mask of FIG. 4, illustrating a positive test result visible through the transparent membrane; and

FIG. 6 depicts a simplified view of a variant of the face mask of FIG. 4.

Like reference numbers and designations in the various drawings indicate like elements.

### DETAILED DESCRIPTION

Before the present methods, implementations, and systems are disclosed and described, it is to be understood that this invention is not limited to specific synthetic methods, specific components, implementation, or to particular compositions, and as such may, of course, vary. It is also to be understood that the terminology used herein is for the purpose of describing particular implementations only and is not intended to be limiting.

As used in the specification and the claims, the singular forms “a, “an” and “the” include plural references unless the context clearly dictates otherwise. Ranges may be expressed in ways including from “about” one particular value, and/or to “about” another particular value. When such a range is expressed, another implementation may include from the one particular value. Another implementation may include from the one particular value and/or the other particular value. Similarly, when values are expressed as approximations, for example by use of the antecedent “about,” it will be understood that the particular value forms another implementation. It will be further understood that the description includes instances where said event or circumstance occurs and instances where it does not.

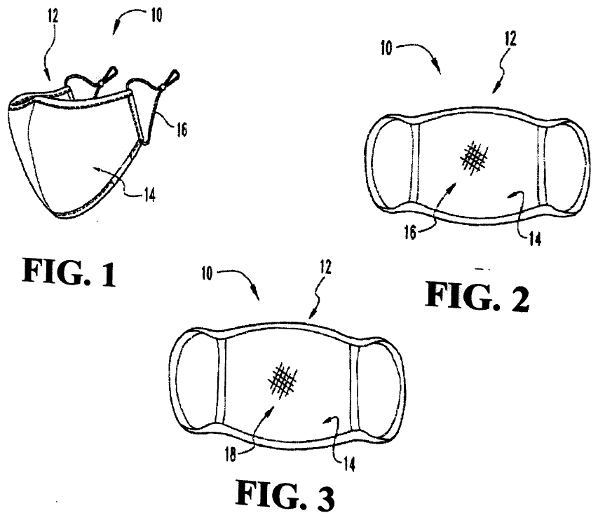

The present disclosure relates generally to a rapid testing mechanism for respiratory viral pathogens. As shown in FIG. 1, an exemplary mechanism for facilitating the rapid testing may include a substrate housing structure 10, such as a face mask 12. The face mask 12, according to one embodiment, may comprise a material, such as filter material 14, straps/ear loops 16 to secure proper positioning of the face mask 12 on the face of a wearer, and a nose wire, which may also be used for positioning.

Typically, the filter material 14 is made up of multiple layers of material. For example, the filter material 14 may be a three-ply material including a melt-blown polymer, such as polypropylene, polyethylene, or vinyl, between non-woven fabric. Numerous factors, such as, for example, shape, size, thickness, number of layers, materials used, fit, breathability, filtering capabilities, disposability, etc. may all be considered, and may vary depending on the application. Additional layers may provide more filtration; however, different materials provide different filtering. Various alternatives to face masks 12 may also be used in combination with the teachings of the present disclosure.

The face mask 12, or an alternative, may be positioned to capture exhaled breath particles from a respiratory tract of a mammal. For example, the face mask 12 may be positioned to cover the mouth and nose of the mammal and capture breath particles in one or more layers of the filter material 14. According to an exemplary embodiment, a layer closest to the source of the exhaled breath particles may be a filter. Some household items may work as a filter layer in a homemade mask, including, for example, paper products that you can breathe through, such as coffee filters, paper towels, and toilet paper. As described above, various structures, materials and configurations may be incorporated into the present disclosure.

Knowing the average size of a virus is about 20-400 nanometers (0.02-0.04 microns), the filter material 14 may capture particles greater than about 400 nanometers (0.4 micrometers). This may allow viruses or viral particles to pass through the filter 14. For example, the novel coronavirus is approximately 0.12 micrometers, so the novel coronavirus would pass freely through the filter material 14, but a bacteria that is 0.2 micrometers would be stopped by the filter material 14. However, most viral particles do not travel independently, but are carried by larger media, such as water droplets, that would be stopped by the filter. This filtering method is provided for exemplary purposes only and other filtering methods may be used. Further, one or more of the filtering methods may be implemented to narrow the viral pathogens that are detected.

At least a portion of the filter material 14 and/or another layer and/or another material is impregnated with or includes a pathogen binding adsorptive reagent. According to exemplary embodiments, the pathogen binding adsorptive reagent may be heparin Sepharose or sulfated cellulose, which may have a pore size of ≥0.4 micron. Although the amounts of the reagent may vary, according to an exemplary embodiment wherein the reagent is a liquid, the filter material 14 may be impregnated with 0.3 mL of the pathogen binding adsorptive reagent.

According to some embodiments, heparin-based porous adsorptive beads are combined with glycerine to slow the drying and extend viability. Further, the housing structure for the filter may be packaged in a porous polymeric membrane.

When the face mask 12 is analyzed, if the binding adsorptive reagent reacts, a positive test for the respiratory viral pathogens is indicated by the filter material 14, as shown at 16 in FIG. 2. According to the exemplary embodiment, the indicator may be a particular color for a positive test. However, various different indicators may be used.

If the pathogen binding reagent does not react, a negative test for respiratory viral pathogens is indicated by the filter material 14, as shown in FIG. 3. For example, a color different from the color indicating a positive test, may be used to indicate a negative test. In some cases, a negative test will be indicated when no change is detected.

In addition to the filtering, the disclosure utilizes affinity chromatography to signify the presence of a virus or viral particles, regardless of the speciation. Non-viral material that is less than 400 nanometer may pass through the filter but will not react with the reagent. This is a low cost, highly sensitive, and qualitative test that is not labor intensive, not prone to operator variation (i.e., correct placement of nasopharyngeal swabs), or reliant on expensive, advanced technology. This technology could be available for home or commercial testing or healthcare point of care testing. The design would allow it to be deployed in resource poor countries. This test may have other novel implications, such as screening mammals (humans or animals) for the presence of contagious diseases before boarding aircraft, within closed spaces, or other places where there might be an increased risk of disease transmission, irrespective of a pandemic state. As stated above, the filter is not restricted to use with a face mask. For example, the filter may be positioned within an aircraft, classroom, office etc. to detect a presence of the virus.

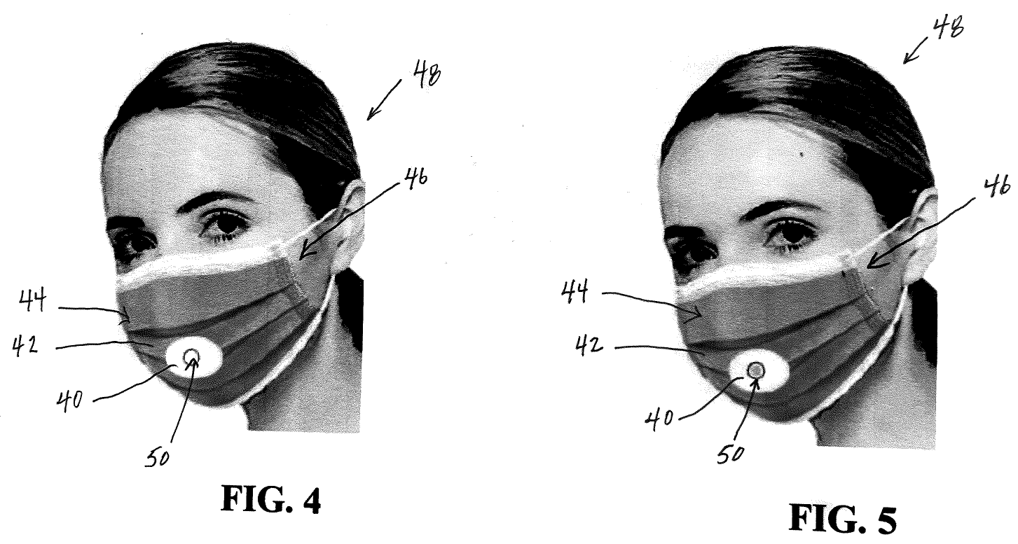

According to another exemplary embodiment, shown in FIG. 4, a transparent membrane 40 may be incorporated into, and visible through, an external layer 42 of the filter material 44 of the mask. As described in the alternative embodiment above, when exhaled breath particles pass through the filter material 44, following a path through the internal layer (opposite the external layer 42), the pathogen binding reagent, and the external layer 42, including the transparent membrane 40, the following occurs:
when the pathogen binding adsorptive reagent reacts, a positive test for respiratory viral pathogens is indicated by the filter material 44; and
when the pathogen binding adsorptive reagent does not react, a negative test for respiratory viral pathogens is indicated by the filter material 44.

According to the present disclosure, when the pathogen binding adsorptive reagent reacts, the positive test indication or the negative test indication is visible to the external environment (i.e., external to the face mask and the wearer 48 of the face mask 46) and through the transparent membrane 40, which is exposed through and/or at the external layer 42.

For example, the transparent membrane 40 can be placed in the exterior layer 42 of the mask (or other test housing structure) or the exterior layer of the porous packet, described above, that contains the pathogen binding reagent. The transparent membrane 40 is typically held in place by heat sealing but can be held in place by a variety of methods including tape adhesive, glue adhesive, sewing, or other methods that fasten two materials together.

Typically, the transparent membrane 40 is composed of polyethylene but may be composed of any semi permeable or nonpermeable translucent or transparent material including, but not limited to, polypropylene, copolyester, acrylic, silicone, cyclic olefin copolymers, or glass. The transparent membrane 40, or other material, may vary in size and configuration to meet the needs of the particular implementation.

The positive and negative test indications may be one or more colors or lack of a color, becoming definitive of the test results after a certain period of time. Note that in FIG. 4 a lack of color, at 50, indicates a negative test result. Although color is used to relay positive and negative test results, it should be appreciated that a variety of different indications could be used. Further, a material other than a transparent membrane 40 may be used.

In FIG. 5, the existence of color, at 50, is visible through the transparent membrane 40 and indicates a positive test result. Color is the primary indicator. Other indicators could include certain shapes, sounds, or light, such as light emitting diodes in the form of a LED display or individual diodes.

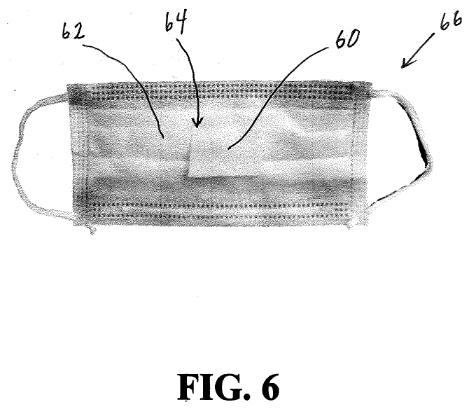

As shown in FIG. 6, a cover/flap 60 may be repositionable over the transparent membrane 40 of the facemask, and may have a first position in which the transparent membrane 40 is covered and a second position in which the transparent membrane 40 is exposed. The cover/flap 60 may be positioned over or within an external layer 62 of the filter material 64 of the mask 66 and may be composed of polypropylene, but could be composed of other fabrics or thermoplastic polymers such as polystyrene, polycarbonate, polyethylene, or polyester. And, depending on the housing type, could be composed of other materials such as paper, cardboard, plastic or metal. The cover/flap 60 may have a flapped or hinged attachment, or other attachment, to the facemask described herein.

The embodiment described with respect to FIG. 6 depicts a means for easy viewing of the positive and negative test results from outside the mask. This may help remedy any issues with only being able to view the positive or negative test results from inside the mask. The cover/flap 60 provides a means for controllably concealing test results to enhance wearer privacy and minimize public display of test results.

In a scenario where different specific viruses are being tested for (e.g., SARS-CoV-2, Influenza, and RSV, a plurality of different indicators could be utilized to signify the location of the pathogen binding substrate associated with the specific virus.

Particular embodiments of the subject matter have been described. Other embodiments are within the scope of the following claims.

1. A rapid testing mechanism, including:
a filter material positioned to capture exhaled breath particles from a respiratory tract of a mammal;
wherein at least a portion of the filter material is impregnated with a pathogen binding adsorptive reagent, wherein the pathogen binding adsorptive reagent is positioned between an internal layer and an external layer of the filter material; and
a transparent membrane positioned at the external layer of the filter material;
wherein, when exhaled breath particles pass through the filter material, the following occurs:
when the pathogen binding adsorptive reagent reacts, a positive test for respiratory viral pathogens is indicated by the filter material; and
when the pathogen binding adsorptive reagent does not react, a negative test for respiratory viral pathogens is indicated by the filter material;
wherein the positive or negative test indication is visible to the external environment through the transparent membrane.

2. The rapid testing mechanism of claim 1, wherein the transparent membrane is incorporated into the external layer of the filter material.

3. The rapid testing mechanism of claim 1, further including a porous sealed packet containing the pathogen binding adsorptive reagent.

4. The rapid testing mechanism of claim 3, wherein the porous sealed packet is positioned between two layers of multiple layers of the filter material.

5. The rapid testing mechanism of claim 1, wherein the pathogen binding adsorptive reagent is heparin sepharose.

6. The rapid testing mechanism of claim 1, wherein the pathogen binding adsorptive reagent is a sulfated cellulose membrane.

7. The rapid testing mechanism of claim 1, wherein the transparent membrane forms an at least partially non-permeable barrier, filtering viruses from the external environment.

8. The rapid testing mechanism of claim 1, further including a cover having a closed position and an open position relative to the transparent membrane.

9. The rapid testing mechanism of claim 1, including a method of testing for a plurality of different viruses using the rapid testing mechanism of claim 1.
